(19) United States
(12) Patent Application Publication
(10) Pub. No.: US 2015/0196227 A1
(43) Pub. Date: Jul. 16, 2015

(54) Medical Apparatus and Patient Table System Thereof

(71) Applicants: Wei Chen, Wuhan (CN); Zhen gang Fan, Shanghai (CN); Ye Shan Ma, Shanghai (CN); Jia Bao Zhu, Shanghai (CN)

(72) Inventors: Wei Chen, Wuhan (CN); Zhen gang Fan, Shanghai (CN); Ye Shan Ma, Shanghai (CN); Jia Bao Zhu, Shanghai (CN)

(21) Appl. No.: 14/598,544

(22) Filed: Jan. 16, 2015

(30) Foreign Application Priority Data
Jan. 16, 2014 (CN) 201420027427.6

(51) Int. Cl. A61B 5/055; A61G 13/02; A61B 6/04

(52) U.S. Cl. CPC A61B 5/0555; A61B 6/0407; A61G 13/02

(57) ABSTRACT

A patient table system includes a patient table that includes a bed board fixed component, a bed board moving component, a linear barcode and a barcode sensor. The bed board moving component is movably disposed on the bed board fixed component. One of the linear barcode and the barcode sensor is disposed on the bed board fixed component, while the other one is disposed on the bed board moving component. The barcode sensor faces the linear barcode and is separated therefrom. The linear barcode marks the position of the bed board moving component in the direction of movement of the bed board moving component relative to the bed board fixed component.

This application claims the benefit of CN 201420027427.6, filed on Jan. 16, 2014, which is hereby incorporated by reference in its entirety.

### FIELD

The disclosed embodiments relate to an imaging apparatus for medical diagnosis, such as a magnetic resonance apparatus and a patient table system thereof.

### BACKGROUND

The position of the patient table in a magnetic resonance apparatus is detected using an optical incremental rotary encoder to allow the patient table to be moved and positioned. However, when an optical incremental rotary encoder is used to detect the patient table position, it is not possible to detect the absolute position of the patient table. Moreover, it has no power-down memory function, so the zero-point position is found before each start-up, making operation inconvenient. In addition, during use, if the patient table is subjected to vibration in a direction perpendicular to the direction of movement thereof, the measured value will fluctuate or be inaccurate, affecting the reliability of the patient table.

Chinese patent CN1977766 proposes a large field-of-view imaging apparatus of a magnetic resonance imaging (MRI) system. The MRI system includes a frame, a magnet mounted on the frame, a patient table in the magnet, and a receiving coil mounted on the patient table. A movable bed board is mounted on the patient table by a support mechanism and engages in feed motion in the receiving coil relative to the patient table.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the disclosed embodiments may provide a highly reliable patient table system with a power-down memory function, in which the absolute position of the patient table may be detected. To this end, the disclosed embodiments provide a patient table system including a patient table. The patient table includes a bed board fixed component, a bed board moving component, a linear barcode and a barcode sensor. The bed board moving component is movably disposed on the bed board fixed component. One of the linear barcode and the barcode sensor is disposed on the bed board fixed component, while the other one is disposed on the bed board moving component. The barcode sensor faces the linear barcode and is separated therefrom. The linear barcode marks the position of the bed board moving component in the direction of movement of the bed board moving component relative to the bed board fixed component.

According to one embodiment, the linear barcode is disposed at the bottom of the bed board moving component and extends in the direction of movement of the bed board moving component relative to the bed board fixed component. The barcode sensor is disposed on the bed board fixed component.

According to one embodiment, the patient table also includes a bed body support. The bed board fixed component is disposed on the bed body support. The barcode sensor is disposed at the end of the bed board fixed component that is close to (or proximate) the bed body support.

According to one embodiment, the patient table system also includes a drive (driving mechanism (or means)), a transmission (transmission mechanism (or means)), a drive driver (driving mechanism (or means) driver) and a controller. The driving mechanism is connected to the bed board moving component by the transmission mechanism. The driving mechanism driver and the controller control the operation of the driving mechanism according to position signals fed back by the barcode sensor.

According to one embodiment, the driving mechanism is an electric motor, the driving mechanism is a transmission chain, a transmission belt or a gear transmission structure, and the driving mechanism driver is an electric motor driver.

According to one embodiment, the linear barcode uniquely marks the position of the bed board moving component in the direction of movement of the bed board moving component relative to the bed board fixed component.

The disclosed embodiments also provide a medical apparatus including the patient table system.

The medical apparatus and patient table system thereof may be useful in the following ways:

1) The linear barcode is used to uniquely mark the position of the bed board moving component in the direction of movement of the bed board moving component relative to the bed board fixed component. For example, each value of the linear barcode corresponds to one position of the bed board moving component. Therefore the value of the linear barcode may be read by the barcode sensor so as to obtain an absolute position of the bed board moving component.

2) Since the linear barcode uniquely marks the position of the bed board moving component in the direction of movement of the bed board moving component relative to the bed board fixed component, operators may know the position of the bed board moving component from the value of the linear barcode read by the barcode sensor even if the power is cut off and then restored. Consequently, there is a power-down memory function for convenient operation.

3) Since the barcode sensor may tolerate movement of the linear barcode within a certain range in a direction perpendicular to the direction of movement of the patient table, no fluctuation or inaccuracy will occur in the measured values even if the patient table is subjected to vibration in a direction perpendicular to the direction of movement of the patient table, making the patient table more reliable. This may also lower the precision requirements for installation, so that installation is easier.

4) Since the barcode sensor faces the linear barcode and is separated therefrom, and reads in a contactless fashion a value of the linear barcode, no friction will occur, enabling reliable operation for a long period of time.

5) Since the barcode sensor is not ferromagnetic, it will not cause deformation of magnetic measuring materials (e.g., a pull cord) in a magnetic field environment, providing (e.g., ensuring) the precision of measurement.

6) The ability of the barcode sensor to distinguish subdivisions of the linear barcode further enables precise positioning and increased measurement precision.

### BRIEF DESCRIPTION OF THE DRAWINGS

FIG. 1 is a schematic diagram of a patient table system in accordance with one embodiment.

FIG. 2 is a schematic diagram of the patient table of FIG. 1.

### DETAILED DESCRIPTION

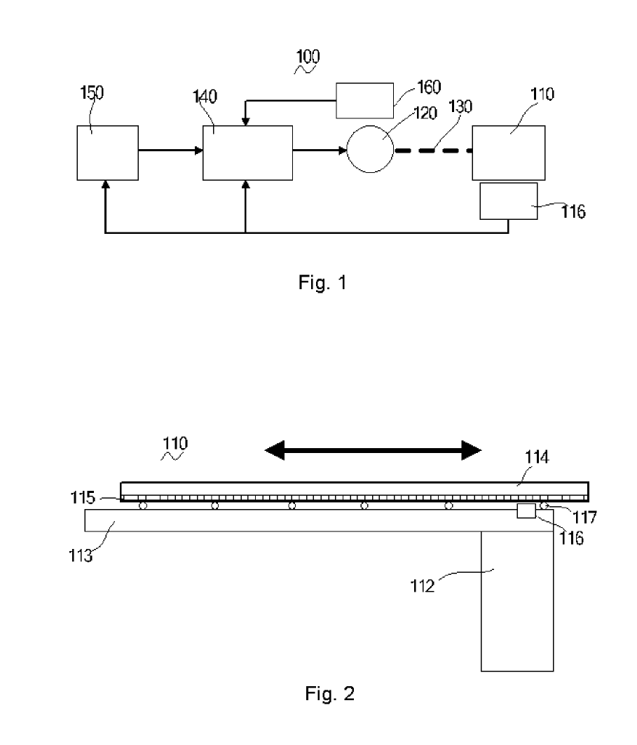

FIG. 1 shows a schematic diagram of the patient table system in accordance with one embodiment. As FIG. 1 shows, the patient table system 100 includes a patient table 110, a driving mechanism (or means) 120, a transmission mechanism (or means) 130, a driving mechanism (or means) driver 140 and a controller 150. The driving mechanism 120 is connected to the patient table 110 by the transmission mechanism 130 and may drive the patient table 110 to move. The driving mechanism driver 140 and controller 150 may control the operation of the driving mechanism 120 according to position signals fed back by the patient table 110, so as to control the position of the patient table 110.

As FIG. 2 shows, the patient table 110 includes a bed body support 112, a bed board fixed component 113, a bed board moving component 114, a linear barcode 115 and a barcode sensor 116.

The bed body support 112 may be used to support and mount the bed board fixed component 113.

The bed board fixed component 113 may be mounted on the bed body support 112. In the embodiment shown in FIG. 2, the bed board fixed component 113 is substantially horizontal.

The bed board moving component 114 is movably disposed on the bed board fixed component 113. For example, the bed board moving component 114 may be movably disposed on the bed board fixed component 113 via rollers 117 (as shown in FIG. 2) or guide rails, etc., and is connected to the driving mechanism 120 by the transmission mechanism 130.

One of the linear barcode 115 and barcode sensor 116 may be disposed on the bed board fixed component 113, while the other one of the linear barcode 115 and barcode sensor 116 may be disposed on the bed board moving component 114. In the embodiment shown in FIG. 2, the linear barcode 115 may be disposed at the bottom of the bed board moving component 114, and extend substantially in the direction of movement of the bed board moving component 114 relative to the bed board fixed component 113 (as indicated by the arrow in FIG. 2). The barcode sensor 116 may be disposed on the bed board fixed component 113, for instance, at the end of the bed board fixed component 113 that is close to (or proximate) the bed body support 112. The barcode sensor 116 faces the linear barcode 115 and is separated therefrom, and reads in a contactless fashion a value of the linear barcode 115 at a position opposite the barcode sensor 116.

The linear barcode 115 may uniquely mark with a number the position of the bed board moving component 114 in the direction of movement of the bed board moving component 114 relative to the bed board fixed component 113. In other words, each value of the linear barcode 115 corresponds to a respective position of the bed board moving component 114. The barcode sensor 116 reads a value of the linear barcode 115 at a position opposite the barcode sensor 116, to obtain an absolute position of the bed board moving component 114.

In one embodiment, the driving mechanism 120 may be an electric motor with a speed sensor 160. The transmission mechanism 130 may be a transmission chain, a transmission belt or a gear transmission structure, etc. The driving mechanism driver 140 may be an electric motor driver.

The structure of the patient table system 100 in accordance with one embodiment is described above. The method of use of the patient table system 100 is outlined below.

In use, a movement position of the controller 150 may be preset externally or by a program. The controller 150 sends a designated position instruction to the driving mechanism driver 140. The driving means driver 140 may start up the driving mechanism 120 according to a preset acceleration and, in accordance with a preset speed, control the driving mechanism 120 to make the bed board moving component 114 move relative to the bed board fixed component 113 in a direction indicated by the arrow in FIG. 2, via the transmission means 130. In the course of this process, the barcode sensor 116 reads the values of the linear barcode 115 in real time and feeds the read values back to the driving mechanism driver 140 and controller 150, thereby enabling the bed board moving component 114 to be moved to the desired position precisely. Furthermore, the driving mechanism 120 may, via a speed sensor thereof, feed the speed of the driving mechanism 120 back to the driving mechanism driver 140, so as to move the bed board moving component 114 to the desired position more precisely. In the above process, even if the driving mechanism driver 140 is powered down, the controller 150 may read the value of the linear barcode 115 via the barcode sensor 116, so that a worker may know the position of the bed board moving component 114 even when the driving means driver 140 is powered down, in order to continue with subsequent operations.

As stated above, the patient table system 100 in accordance with one embodiment may be useful in the following ways:

1) The linear barcode 115 is used to uniquely mark the position of the bed board moving component 114 in the direction of movement of the bed board moving component 114 relative to the bed board fixed component 113. Each value of the linear barcode 115 corresponds to a respective position of the bed board moving component 114. Therefore the value of the linear barcode 115 may be read by the barcode sensor 116 so as to obtain an absolute position of the bed board moving component 114.

2) Since the linear barcode 115 uniquely marks the position of the bed board moving component 114 in the direction of movement of the bed board moving component 114 relative to the bed board fixed component 113, the controller 150 may read the value of the linear barcode 115 via the barcode sensor 116 even if the power is cut off and then restored, so that workers know the position of the bed board moving component 114. Consequently, there is a power-down memory function, for convenient operation.

3) Since the barcode sensor 116 may tolerate movement of the linear barcode 115 within a certain range in a direction perpendicular to the direction of movement of the patient table 110, no fluctuation or inaccuracy will occur in the measured values even if the patient table 110 is subjected to vibration in a direction perpendicular to the direction of movement of the patient table 110, making the patient table 100 more reliable. This may also lower the precision requirements for installation, so that installation is easier.

4) Since the barcode sensor 116 faces the linear barcode 115 and is separated therefrom, and reads in a contactless fashion a value of the linear barcode 115, no friction will occur, enabling reliable operation for a long period of time.

5) Since the barcode sensor 116 is not ferromagnetic, it will not cause deformation of magnetic measuring materials (e.g., a pull cord) in a magnetic field environment, ensuring the precision of measurement.

6) The ability of the barcode sensor 116 to distinguish subdivisions of the linear barcode 115 further enables precise positioning and increased measurement precision.

The disclosed embodiments also provide a medical apparatus using the patient table system 100 described above. For example, the medical apparatus may be a magnetic resonance apparatus, a CT apparatus, a molecular imaging apparatus, or an X-ray machine, etc. Since the patient table system will not cause deformation of other components in a magnetic field, it is suitable for use in a magnetic resonance apparatus.

Disclosed is a patient table system, including a patient table. The patient table includes a bed board fixed component, a bed board moving component, a linear barcode and a barcode sensor. The bed board moving component is movably disposed on the bed board fixed component. One of the linear barcode and the barcode sensor is disposed on the bed board fixed component, while the other one is disposed on the bed board moving component. The barcode sensor faces the linear barcode and is separated therefrom. The linear barcode marks the position of the bed board moving component in the direction of movement of the bed board moving component relative to the bed board fixed component. As the linear barcode uniquely marks the position of the bed board moving component in the direction of movement of the bed board moving component relative to the bed board fixed component, the absolute position of the bed board moving component may be obtained using the barcode sensor 116, and there is a power-down memory function.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A patient table system comprising:
a patient table comprising a bed board fixed component, a bed board moving component, a linear barcode, and a barcode sensor;
wherein:
the bed board moving component is movably disposed on the bed board fixed component;
one of the linear barcode and the barcode sensor is disposed on the bed board fixed component, while the other one of the linear barcode and the barcode sensor is disposed on the bed board moving component; and
the barcode sensor faces the linear barcode and is separated from the linear barcode.

2. The patient table system of claim 1, wherein the linear barcode is disposed at a bottom of the bed board moving component and extends in a direction of movement of the bed board moving component relative to the bed board fixed component, and wherein the barcode sensor is disposed on the bed board fixed component.

3. The patient table system of claim 2, wherein:
the patient table further comprises a bed body support;
the bed board fixed component is disposed on the bed body support; and
the barcode sensor is disposed at an end of the bed board fixed component that is proximate the bed body support.

4. The patient table system of claim 1, wherein:
the patient table system further comprises a driving mechanism, a transmission mechanism, a driving mechanism driver and a controller;
the driving mechanism is connected to the bed board moving component by the transmission mechanism; and
the driving mechanism driver and the controller control the operation of the driving mechanism according to position signals fed back by the barcode sensor.

5. The patient table system of claim 4, wherein:
the driving mechanism is an electric motor;
the driving mechanism is a transmission chain, a transmission belt, or a gear transmission structure; and
the driving mechanism driver is an electric motor driver.

6. The patient table system of claim 1, wherein the linear barcode marks the position of the bed board moving component in a direction of movement of the bed board moving component relative to the bed board fixed component.

7. The patient table system of claim 1, wherein the linear barcode uniquely marks a position of the bed board moving component in a direction of movement of the bed board moving component relative to the bed board fixed component.

8. A medical apparatus comprising a patient table system, the patient table system comprising:
a patient table comprising a bed board fixed component, a bed board moving component, a linear barcode, and a barcode sensor;
wherein:
the bed board moving component is movably disposed on the bed board fixed component;
one of the linear barcode and the barcode sensor is disposed on the bed board fixed component, while the other one of the linear barcode and the barcode sensor is disposed on the bed board moving component; and
the barcode sensor faces the linear barcode and is separated from the linear barcode.

9. The medical apparatus of claim 8, wherein the linear barcode is disposed at a bottom of the bed board moving component and extends in a direction of movement of the bed board moving component relative to the bed board fixed component, and wherein the barcode sensor is disposed on the bed board fixed component.

10. The medical apparatus of claim 9, wherein:
the patient table further comprises a bed body support;
the bed board fixed component is disposed on the bed body support; and
the barcode sensor is disposed at an end of the bed board fixed component that is proximate the bed body support.

11. The medical apparatus of claim 8, wherein:
the patient table system further comprises a driving mechanism, a transmission mechanism, a driving mechanism driver and a controller;
the driving mechanism is connected to the bed board moving component by the transmission mechanism; and
the driving mechanism driver and the controller control the operation of the driving mechanism according to position signals fed back by the barcode sensor.

12. The medical apparatus of claim 11, wherein:
the driving mechanism is an electric motor;
the driving mechanism is a transmission chain, a transmission belt, or a gear transmission structure; and
the driving mechanism driver is an electric motor driver.

13. The medical apparatus of claim 8, wherein the linear barcode marks the position of the bed board moving component in a direction of movement of the bed board moving component relative to the bed board fixed component.

14. The medical apparatus of claim 8, wherein the linear barcode uniquely marks a position of the bed board moving component in a direction of movement of the bed board moving component relative to the bed board fixed component.
